(19) United States
(12) Patent Application Publication
(10) Pub. No.: US 2007/0286832 A1
(43) Pub. Date: Dec. 13, 2007

(54) Multi-phase personal cleansing compositions comprising two aqueous phases

(76) Inventors: Mannie Lee Clapp, Mason, OH (US); Edward Dewey Smith, Mason, OH (US); Rebecca Ann Taylor, Cincinnati, OH (US); Jimmie Lee Ward, Middletown, OH (US); Karen Lehnhoff, Cincinnati, OH (US); Christopher Dean Putman, West Chester, OH (US); Mark Robert Sivik, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/451,085

(22) Filed: Jun. 12, 2006

(51) Int. Cl. A61K 8/81

(52) U.S. Cl. 424/70.11; 424/70.16

(57) ABSTRACT

The present invention relates to a multi-phase personal-cleansing composition comprising a first aqueous phase and a second aqueous phase packaged in physical contact with one another. The first aqueous phase comprises a surfactant, a salt and water. The second aqueous phase comprises a polymer, a salt and water. The molality of the salt of the first aqueous phase differs from that of the salt of the second aqueous phase by less than 6%. The weight percent of water in the first aqueous phase differs by at least 5% from that of the second aqueous phase. The viscosity of the first aqueous phase differs from that of the second aqueous phase by less than about 25%. Preferably, the polymer in the second aqueous phase is a high solution viscosity polymer having a 1% viscosity at pH 7 of greater than 750 centipose.

### FIELD OF THE INVENTION

The present invention relates to a multi-phase personal cleansing composition comprises a first aqueous phase that comprises a surfactant and a second aqueous phase that comprises a polymer, the first aqueous phase and the second aqueous phase being packaged in physical contact.

### BACKGROUND OF THE INVENTION

Consumers continue to desire multi-phase personal cleansing compositions which are both eye-catching on the store-shelf and deliver multiple benefits. One method of providing such a personal cleansing product is to provide a multiphase product in which the phases are packaged in physical contact with each other, so that the multiphase nature of the product is visible to the consumer, and the phases are co-dispensed by the consumer at the time of use.

Maintaining the stability of the multiphase compositions with dissimilar aqueous phases in physical contact with each other for any period of time has proved to be a problem. The physical contact of two phases with different water concentrations often result in the water concentrations of each phase changing as water molecules equilibrate across the interface of the two phases. The exchange of water concentrations between the two phases lead to a multi-phase personal cleansing composition which has unacceptable skin feel, poor dispensing characteristics and unacceptable visual appeal to consumers. For example, the multiphase personal cleansing compositions have a tendency to become clumpy, curdled, or coagulated during dispensing and fall off the puff or applicator during application.

One method of providing a multiphase aqueous/aqueous product while maintaining stability would be the use of dual-chamber packaging. These packages could comprise separate aqueous compositions, and allow for the co-dispensing of the two in a single or dual stream. The separate compositions thus remain physically separate and stable during prolonged storage and just prior to application, but then mix during or after dispensing. Although such dual-chamber delivery systems provide improved performance benefits versus conventional systems, it is often difficult to achieve consistent and uniform performance because of the uneven dispensing ratio between the two phases from these dual-chamber packages. Additionally, these packaging systems add considerable cost to the finished product.

Accordingly, the need still remains for multiphase personal cleansing compositions that provide cleansing with increased lather longevity and improvements in lathering characteristics, skin feel, dispensing characteristics and appearance during and after dispensing.

### SUMMARY OF THE INVENTION

It has now been found that multi-phase personal cleansing compositions comprising two dissimilar aqueous phases can be formulated remain stable and osmotically balanced when packaged in physical contact with one another.

The present invention relates to a multi-phase personal-cleansing composition comprising a first aqueous phase and a second aqueous phase packaged in physical contact with one another. The first aqueous phase comprises a surfactant, a salt and water. The second aqueous phase comprises a polymer, a salt and water. The molality of the salt of the first aqueous phase differs from that of the salt of the second aqueous phase by less than 6%. The weight percent of water in the first aqueous phase differs by at least 5% from that of the second aqueous phase. The viscosity of the first aqueous phase differs from that of the second aqueous phase by less than about 25%. Preferably, the polymer in the second aqueous phase is a high solution viscosity polymer having a 1% viscosity at pH 7 of greater than 750 centipoise.

The multi-phase personal cleansing compositions of the present invention comprise a first aqueous phase that comprises a surfactant and a second aqueous phase comprising a polymer that are packaged in physical contact yet remain stable. The stability is due to the careful balancing of polymer type and polymer level to provide for an osmotic pressure in the second aqueous phase comprising polymer which is equal to the osmotic pressure first aqueous phase comprising surfactant. When this osmotic pressure balance is reached, the net water transport across the interface is effectively zero.

The compositions of the present invention further provide superior aesthetics in the package via the multi-phased appearance and improved skin feel during and after application. Moreover, the compositions of the present invention have a more uniform appearance during dispensing and appear less clumpy, curdled, or coagulated.

### DETAILED DESCRIPTION OF THE INVENTION

The term “ambient conditions” as used herein, refers to surrounding conditions at one (1) atmosphere of pressure, 50% relative humidity, and 25° C.

The term “molality” refers to the number of moles of solute per kilogram of solvent. For example, a 7.5%, by weight of the phase, of salt in a surfactant formula which is 65%, by weight of the phase, of water would be (7.5/58.4) (=grams in formula/molecular weight)=0.13 moles of salt in 0.065 kg water=1.98 molal.

By the term “multi-phased” or “multi-phase” as used herein, is meant that the first aqueous phase and the second aqueous phase herein occupy separate but distinct physical spaces inside the package in which they are stored, but are in direct contact with one another (i.e., they are not separated by a barrier and they are not emulsified or mixed to any significant degree). In one preferred embodiment of the present invention, the “multi-phase” personal cleansing compositions comprising the first aqueous phase and the second aqueous phase are present within the container as a visually distinct pattern. The pattern results from the mixing or homogenization of the “multi-phased” composition. The patterns include but are not limited to the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. The pattern may be striped and may be relatively uniform and even across the dimension of the package. Alternatively, the striped pattern may be uneven, i.e. wavy, or may be non-uniform in dimension. The striped pattern does not need to necessarily extend across the entire dimension of the package. The size of the stripes is at least about 0.1 mm in width and 10 mm in length, preferably at least about 1 mm in width and at least 20 mm in length. The phases can form various geometric shapes, be various different colors, or include glitter or pearlescence.

The term “osmotically balanced” from a water content standpoint indicates that the first aqueous phase has a percent water weight change of less than 5% after storage in physical contact with the second aqueous phase for 7 days according to the Dialysis Method, described in detail below.

The term “osmotic pressure” as used herein is the force exerted by water on a phase of a multiphase composition. Water has a tendency to flow from one aqueous phase to another aqueous phase in a multiphase composition from regions of high water concentrations to regions of lower water concentration through the interface between the two aqueous phases. Osmotic pressure is controlled by both the materials which comprise a multi-phase personal cleansing composition and the concentrations of those materials in a phase within the multi-phase personal cleansing composition.

The term “personal cleansing composition” as used herein, refers to compositions intended for topical application to the skin or hair. These can composition may also be used for shaving aids.

The “percent difference” as used herein, whether it be for the molality of salt, the weight percent of water, or viscosities between two phases is calculated by dividing the smallest numerical value by the largest numerical value, multiplying by one hundred percent (100%), and then subtracting the resultant value from 100% to get the percent difference.

The term “phases” as used herein, refers to a region of a composition having one average composition, as distinct from another region having a different average composition, wherein the regions are visible to the naked eye. This would not preclude the distinct regions from comprising two similar phases where one phase could comprise pigments, dyes, particles, and various optional ingredients, hence a region of a different average composition.

The term “stable” as used herein, unless otherwise specified, refers to compositions that maintain at least two “separate” phases when sitting in physical contact at ambient conditions for a period of at least about 180 days. By “separate” is meant that there is substantially no mixing of the phases, observable to the naked eye, and prior to dispensing of the composition.

The phrase “substantially free of” as used herein, means that the composition comprises less than about 3%, preferably less than about 1%, more preferably less than about 0.5%, even more preferably less than about 0.25%, and most preferably less than about 0.1%, by weight of the composition, of the stated ingredient.

The personal cleansing compositions and methods of the present invention can comprise, consist of, or consist essentially of, the essential elements and limitations of the invention described herein, as well as any additional or optional ingredients, components, or limitations described herein or otherwise useful in personal cleansing compositions intended for topical application to the hair or skin.

Product Form: The personal cleansing compositions of the present invention are typically in the form of a liquid. The term “liquid” as used herein means that the composition is generally flowable to some degree. “Liquids”, therefore, can include liquid, semi-liquid, cream, lotion or gel compositions intended for topical application to skin. The compositions typically exhibit a viscosity of equal to or greater than about 3,000 cps to about 1,000,000 cps, as measured by the T-bar Viscosity Method described hereinafter. In addition, the ratio of the first aqueous phase to the second aqueous phase is from about 10:1 to about 1:10.

The compositions comprise a two aqueous phases, both of which are described in greater detail hereinafter. In a preferred embodiment of the present invention the multi-phased personal cleansing composition, the composition has at least two visually distinct phases wherein at least one phase is visually distinct from a second phase. The visually distinct phases are packaged in physical contact with one another and are stable.

The product forms contemplated for purposes of defining the compositions and methods of the present invention are rinse-off formulations, by which is meant the product is applied topically to the skin or hair and then subsequently (i.e., within minutes) rinsed away with water, or otherwise wiped off using a substrate or other suitable removal means.

Salt: The first aqueous phase and second aqueous phase both comprise a salt. In some embodiments, the molality of the salt in the first aqueous phase differs from the molality of the salt in the second aqueous phase by less than about 6%, preferably the molality of the salt of the first aqueous phase differs from that of the salt in the second aqueous phase by less than about 3%, more preferably the molality of the salt of the first aqueous phase differs from that of the salt of the second aqueous phase by less than about 1%, most preferably the molality of the salt of the first aqueous phase differs from the salt of the second aqueous phase by less than about 0.5%. The difference in molality of the salt of the first aqueous phase and the salt of the second aqueous phase is calculated by dividing the smallest numerical value of molality by the largest numerical value of molality, multiplying by one hundred percent (100%), and then, subtracting the resultant value from 100% to get the percent difference.

The salt in the first aqueous phase and the salt in the second aqueous phase within the multiphase composition can added per se to the composition or it can be formed in situ via the counter-ions included in one of the raw materials. Often, the surfactant containing phase will contain a significant amount of salt brought in by the surfactants themselves, particularly if they are from the class of amphoteric or zwitterionic surfactants. Any salt in the phase, whether added or brought in via a raw material is included in the molality calculation for the purposes of this invention. The salt in the first aqueous phase and the salt in the second aqueous phase preferably includes an anion comprising phosphate, chloride, sulfate or citrate and a cation comprising sodium, ammonium, potassium, magnesium or mixtures thereof. Some preferred salts are sodium or ammonium chloride or sodium or ammonium sulfate. Preferred salts are sodium chloride and sodium sulfate. The salt in the first aqueous phase and the salt in the second aqueous phase can be the same salt or can be a different salt. Preferably the salt in the first aqueous phase and the salt in the same aqueous phase are the same salt.

The salt is generally present in an amount from about 0.1% by weight to about 15% by weight, preferably from about 1% to about 8% by weight of the first aqueous phase or second aqueous phase, but may be varied if required.

Water: The first aqueous phase and second aqueous phase comprise water as the primary component. The first aqueous phase of the present invention and the second aqueous phase of the present invention can comprise from about 30% to about 99%, by weight of water. The first aqueous surfactant containing phase preferably comprises more than about 20% water, more preferably more than about 40% water, and more preferably more than about 50% and most preferably more than about 60% water. The second aqueous phase generally comprises more than about 50%, preferably more than about 60%, even more preferably more than about 70%, still more preferably more than about 80%, by weight of the second aqueous phase, of water. The weight percent of water in the first aqueous phase differs by at least 5% from the weight percent of water in the second aqueous phase. Preferably, the weight percent of water in the first aqueous phase differs by at least 7% from that in the second aqueous phase. More preferably, the weight percent of water in the first aqueous phase differs by at least 10% from that in the second aqueous phase. For example, when the first aqueous phase comprises 50%, by weight of the first aqueous phase, of water and the second aqueous phase comprises 70%, by weight of the second phase of water then the difference is 20% by weight.

The typical multiphase composition of the present invention will typically have a pH of from about 5 to about 8, more preferably from about 5 to about 6.5. The first aqueous phase or second aqueous phase can optionally comprise a pH regulator to facilitate the proper pH range. Preferably, the pH of the first aqueous phase will be within ±0.25 pH units of the second aqueous phase.

Viscosity of the Phases: The viscosity of the first aqueous phase differs from the viscosity of the second aqueous phase by less than about 25%. It has been found that when the viscosity of the first aqueous phase and the viscosity of the second aqueous phase are similar to each other that the consumers perceive that the overall multi-phase personal cleansing composition has better dispensing and appearance than those multiphase composition whose phases differ greatly in viscosity. In some embodiments, the viscosity of the first aqueous phase differs from that of the second aqueous phase by less than about 15%; more preferably, the viscosity of the first aqueous phase differs from that the second aqueous phase by less than about 10% and most preferably, the viscosity of the first aqueous phase differs from that of the second aqueous phase by less than about 5%. Preferably, the first aqueous phase and second aqueous phase have a viscosity of greater than about 3,000 centipoise (“cps”), more preferably greater than about 10,000 cps, even more preferably greater than about 20,000 cps, and still more preferably greater than about 30,000 cps, as measured by the Viscosity Method described hereinafter. In some embodiments, the first aqueous phase of the multi-phase personal cleansing composition of the present invention comprises a viscosity greater than 30,000 cps. In other embodiments, the first aqueous phase comprises a viscosity greater than the second aqueous phase in the multi-phase personal cleansing composition of the present invention. The difference in viscosities of the first aqueous phase and that of the second aqueous phase is calculated by dividing the smallest numerical value of viscosity by the largest numerical value of viscosity, multiplying by one hundred percent (100%), and then, subtracting the resultant value from 100% to get the percent difference.

The multi-phase personal cleansing compositions of the present invention are osmotically balanced in that the first or second aqueous phase first aqueous phase has a percent water weight change of less than 5% after storage in physical contact with the second aqueous phase for 7 days according to the Dialysis Method, described in detail below. In some embodiments, the first or second aqueous phase first aqueous phase has a percent water weight change of less than 3% after storage in physical contact with the second aqueous phase for 7 days according to the Dialysis Method, described in detail below. In other embodiments, the first or second aqueous phase first aqueous phase has a percent water weight change of less than 1% after storage in physical contact with the second aqueous phase for 7 days according to the Dialysis Method, described in detail below.

First Aqueous Phase: The first aqueous phase comprises a surfactant, water and a salt, as discussed above. The first aqueous phase of the multi-phase personal cleansing compositions can produces a Total Lather Volume of at least about 500 ml, more preferably greater than about 600 ml, even more preferably greater than about 700 ml, even more preferably greater than about 800 ml, still more preferably greater than about 1000 ml, and still even more preferably greater than about 1250 ml as measured by the Lather Volume Test described hereinafter. The first aqueous phase of the multi-phase personal cleansing compositions preferably produces a Flash Lather Volume of at least about 200 ml, preferably greater than about 250 ml, even more preferably greater than about 300 ml, as measured by the Lather Volume Test described hereinafter. Preferably, the first aqueous phase has a Yield Point of greater than about 0.1 Pascal (Pa), more preferably greater than about 1 Pascal, even more preferably greater than about 10 Pascal, and still more preferably greater than about 30 Pascal, as measured by the Yield Point Method described hereinafter.

Surfactant: The first aqueous phase of the present invention comprises a cleansing surfactant suitable for application to the skin or hair. Suitable surfactants for use herein include any known or otherwise effective cleansing surfactant which are suitable for application to the skin, and which are otherwise compatible with the other essential ingredients in the first aqueous or second aqueous phase of the composition. These cleansing surfactants include anionic, nonionic, cationic, zwitterionic or amphoteric surfactants, or combinations thereof. Suitable surfactants are described in McCutcheon's, Emulsifiers and Detergents, 1989 Annual, published by M. C. Publishing Co., and in U.S. Pat. No. 3,929,678 issued to Laughlin, et al. on Dec. 30, 1975.

The first aqueous phase of the multi-phase personal cleansing compositions may comprise a surfactant at concentrations ranging from about ranging from about 2% to about 23.5%, more preferably from about 3% to about 21%, even more preferably from about 4% to about 20.4%, still more preferably from about 5% to about 20%, still even more preferably from about 13% to about 18.5%, and even still even more preferably from about 14% to about 18%, by weight of the first aqueous phase. The pH range of the first aqueous phase may be from about 5 to about 8 or to about 6.5.

Preferred linear anionic surfactants for use in the first aqueous phase of the multi-phase personal cleansing composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in commonly owned U.S. Application Ser. No. 60/680,149 entitled “Structured Multi-phased Personal Cleansing Compositions Comprising Branched Anionic Surfactants” filed on May 12, 2005 by Smith, et al. Branched anionic surfactants include but are not limited to the following surfactants: sodium trideceth sulfate, sodium tridecyl sulfate, sodium C₁₂-₁₃ alkyl sulfate, and C₁₂-₁₃ pareth sulfate and sodium C₁₂-₁₃ pareth-n sulfate.

Anionic surfactants with branched alkyl chains such as sodium trideceth sulfate, for example, may be employed in some embodiments. Mixtures of anionic surfactants can also be used in some embodiments.

Other surfactants from the classes of amphoteric, zwitterionic surfactant, cationic surfactant, and/or nonionic surfactant can be incorporated in first aqueous phase of the compositions.

In addition to the alkylamphoacetates, other amphoteric surfactants are suitable for use in the multiphase composition of the present invention. The amphoteric surfactants include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, and N-alkyltaurines. Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the multi-phase personal cleansing composition include betaines, including cocoamidopropyl betaine. Amphoacetates and diamphoacetates can also be used.

Cationic surfactants can also be used in the first aqueous phase, but are generally less preferred, and thus, may represent less than about 5%, by weight of the first aqueous phase.

Suitable nonionic surfactants for use in first aqueous phase include condensation products of alkylene oxide groups (hydrophilic in nature) with an organic hydrophobic compound, which may be aliphatic or alkyl aromatic in nature.

Lamellar Structurant: In some embodiments, the first aqueous phase is a lamellar. Thus, in some embodiments the first aqueous phase of the present compositions can optionally and preferably, further comprise about 0.1% to 10% by weight of first aqueous phase, of a lamellar structurant which functions in the compositions to form a lamellar phase. It is believed the lamellar phase enhances the interfacial stability between the first aqueous phase comprising surfactant and the second aqueous phase comprising polymer of the present compositions.

Suitable lamellar structurants include a fatty acid or ester derivatives thereof, a fatty alcohol, an ethoxylated fatty alcohol, trihydroxystearin (available from Rheox, Inc. under the trade name THIXCIN® R), or polymethyacrylamidopropyl trimonium chloride (available from Rhodia under the trade name POLYCARE® 133), or an alkanolamide. If the lamellar structurant is a fatty acid, or an ester of fatty acid, the hydrocarbon backbone can be straight chained or branched. Preferably, the lamellar structurant is selected from lauric acid, fatty alcohols, ethoxylated fatty alcohols, or trihydroxystearin.

In one embodiment of the present invention, the first aqueous phase comprises an anionic surfactant (e.g. sodium trideceth sulfate), an amphoacetate surfactant (e.g. sodium lauroamphoacetate), and an ethoxylated fatty alcohol (e.g. TDA-3 from BASF). The first aqueous phase of this embodiment preferably further comprises an electrolyte (e.g. sodium chloride or sodium sulfate)

Second Aqueous Phase: The second aqueous phase of the present invention comprises a polymer, water and a salt, as described above. The second aqueous phase of the present invention comprises less than about 5%, preferably less than about 3%, and more preferably less than about 1%, by weight of the second aqueous phase, of a surfactant. In one embodiment of the present invention, the second aqueous phase is free of surfactant. The second aqueous phase of the multi-phase personal cleansing compositions preferably produces a Total Lather Volume of no greater than about 500 ml, more preferably no greater than about 400 ml, even more preferably no greater than about 350 ml, as measured by the Lather Volume Test described hereinafter. The second aqueous phase of the multi-phase personal cleansing compositions preferably produces a Flash Lather Volume of no greater than about 150 ml, preferably no greater than about 130 ml, even more preferably no greater than about 110 ml, as measured by the Lather Volume Test described hereinafter. Preferably, the second aqueous phase exhibits a Yield Point of at least about 0.1 Pa, preferably at least about 1 Pa, more preferably at least about 10 Pa, as measured by the Yield Point Method described hereinafter.

In some embodiments, the second aqueous phase of the multiphase personal cleansing composition can comprise a weight percent of salt in the second aqueous phase greater than a weight percent of surfactant.

Polymer: The function of the polymer in the second aqueous phase is two-fold, to provide the second aqueous phase with a viscosity similar to first aqueous phase comprising surfactant and to provide for a suitable osmotic pressure to provide for stability against the first aqueous surfactant phase.

The second aqueous phase can be comprised of one or more polymers. The total solution viscosity of the polymer solution must meet the viscosity requirements described above regardless of the number of different polymers in the system. If a high polymer level is needed to provide for the appropriate osmotic pressure then it is advantageous to use polymers of high solution viscosity in combination with those of low solution viscosity.

The multi-phase personal cleansing composition of the present invention preferably comprises a high solution viscosity polymer wherein the polymer has a 1% solution viscosity at pH 7 of greater than 750 centipose (cps), preferably greater than 1000 cps, even more preferably greater than 2000 cps, most preferably greater than 5000 cps. Non-limiting examples of high solution viscosity polymers for use in the composition include Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), cellulosic gel, hydroxypropyl starch phosphate (Structure XL from National Starch) synthetic and natural gums and thickeners such as xanthan gum (Ketrol CG-T from CP Kelco), succinoglycan (Rheozan from Rhodia, gellum gum, pectin, alginates, starches including pregelatinized starches, modified starches, or mixtures thereof.

The low solution viscosity polymers in the present invention should have a 1% viscosity at pH 7 of less than 500 cps. The polymer may be neutral at this pH or ionic, i.e. charged, either cationic or anionic. The polymer may be a homopolymer or a copolymer of more than one monomer. Non-limiting examples of monomers used to make these polymers are acrylamide, methacrylamide, NN-dimethyl-acrylamide, methyl allyl ether, N-Vinyl pyrrolidone, Vinyl Caprolactam, methyl acrylate, methyl methacrylate, dimethyl-amino-ethyl Methacrylate, Dimethyl-amino ethyl acrylate, dimethyl diallyl ammonium chloride, acryalmidopropyl trimethyl ammonium chloride, theacrylamidoprpyl trimtheyl ammonium chloride, quaternized vinyl imidazole, N-carboxymethyl-4-vinylpyridnium chrloride, sodium acrylate, sodium methacrylate, 2-acrylamido-2-methyl propane sulfonate, and ethylene oxide.

The second aqueous phase of the present compositions can further comprise optional ingredients such as those described hereinafter. Preferred optional ingredients for the second aqueous phase include pigments, pH regulators, and preservatives. In one embodiment, the second aqueous phase comprises a polymer (e.g. acrylates/vinyl isodecanoate crosspolymer), water, a pH regulator (e.g. triethanolamine), and a preservative (e.g. 1,3-dimethylol-5,5-dimethylhydantoin (“DMDMH” available from Lonza under the trade name GLYDANT®)).

In some embodiments, the first aqueous phase may comprise the polymers in the amounts described herein for the second aqueous phase. In some embodiments, the second phase of the multi-phase personal cleansing composition may comprise a weight percent of polymer that is less than the weight percent of salt of the second aqueous phase. In some embodiments, the first phase of the multi-phase personal cleansing composition has a lesser amount of polymer than the second aqueous phase.

Colorant: In a preferred embodiment the multiphase personal cleansing composition comprises a colorant in at least one phase of the multiphase personal cleansing composition. The composition comprises from about 0.00001% to about 10%, by weight of the composition of a colorant. Preferably, the multi-phase personal cleansing compositions comprises from about 0.0001% to about 1%, more preferably from about 0.001% to about 0.1%, even more preferably from about 0.005% to about 0.05%, by weight of the composition of a colorant. Some colorants useful in the multiphase personal cleansing composition are described in the co-pending application U.S. application Ser. No. 11/304831 entitles “Non-migrating colorants in multi-phase personal cleansing compositions” filed on Dec. 15, 2005. The colorants for use in the multi-phase personal cleansing compositions are selected from the group consisting of organic pigments, inorganic pigments, interference pigments, lakes, natural colorants, pearlescent agents, dyes, carmines, and mixtures thereof. The colorant, in a preferred embodiment, comprises metal ions. Preferably, the colorant is free of barium and aluminum ions which allows for improved lamellar phase stability. The colorant preferably maintains UV stability.

Optional Ingredients: A variety of suitable optional ingredients can be employed in the first aqueous phase and the second aqueous phase. Non-limiting optional ingredients include humectants and solutes. A variety of humectants and solutes can be employed and can be present at a level of from about 0.1% to about 50%, preferably from about 0.5% to about 35%, and more preferably from about 2% to about 20%, by weight of the multi-phase personal cleansing composition. Preferred humectants are glycerin and sorbitol.

Suitable optional ingredients further include skin conditioning agents. Nonionic polyethylene/polypropylene glycol polymers (Polyox from Dow Chemical) are preferably used as skin conditioning agents. In the compositions of the present invention, the compositions may comprise less than 4%, preferably less than 2%, more preferably less than 1%, and most preferably less than 0.5%, by weight of the multiphase personal cleansing composition of a polyalkylene glycol.

The multi-phase personal cleansing compositions of the present invention can additionally comprise an organic cationic deposition polymer in the first aqueous phase or the second aqueous phase. Suitable cationic deposition polymers for use in the compositions of the present invention contain cationic nitrogen-containing moieties such as quaternary ammonium moieties. A non-limiting example of a commercially available synthetic cationic polymer for use in the cleansing compositions is polymethyacrylamidopropyl trimonium chloride, available under the trade name Polycare 133, from Rhodia, Cranberry, N.J., U.S.A.

Other non-limiting examples of these optional ingredients include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the antimicrobial integrity of the cleansing compositions (e.g., DMDMH); anti-acne medicaments (resorcinol, salicylic acid, and the like); antioxidants; skin soothing and healing agents (i.e. aloe vera extract, allantoin); chelators; sequestrants; and agents suitable for aesthetic purposes (i.e. fragrances, essential oils, skin sensates, particles, particles or beads, exfoliating beads pigments, pearlescent agents (e.g., mica and titanium dioxide)) and the like (e.g., clove oil, menthol, camphor, eucalyptus oil, and eugenol). These materials can be used at ranges sufficient to provide the required benefit, as would be obvious to one skilled in the art.

To the extent any optional ingredients described herein include specific materials described hereinbefore as water structurants or lamellar structurants, such materials shall be considered water structurants or lamellar structurants for the purposes of the present invention.

### Test Methods:

T-Bar Viscosity Method: The viscosity can be assessed in first aqueous phase and second phase by the T-Bar Viscosity Method. The apparatus for T-Bar measurement includes a Brookfield DV-II+ Pro Viscometer with Helipath Accessory; chuck, weight and closer assembly for T-bar attachment; a T-bar Spindle D, a personal computer with Rheocalc software from Brookfield, and a cable connecting the Brookfield Viscometer to the computer. First, weigh 80 grams of the first or second aqueous phase in a 4-oz glass jar. Measure the T-bar viscosity by carefully dropping the T-Bar Spindle to the interior bottom of the jar and set the Helipath stand to travel in an upward direction. Open the Rheocalc software and set the following data acquisition parameters: set Speed to 5 rpm, set Time Wait for Torque to 00:01 (1 second), set Loop Start Count at 100. Start data acquisition and turn on the Helipath stand to travel upward at a speed of 22 mm/min. The T-Bar viscosity “T,” is the average T-Bar viscosity reading between the 6^{th} reading and the 95^{th} reading (the first five and the last five readings are not used for the average T-Bar viscosity calculation). If the viscosity is below the lower limit of the D spindle (30,000 cps), a larger spindle can be used for the T-Bar Viscosity measurement.

Lather Volume Test: Lather volume of a multi-phase personal cleansing composition or of a second aqueous phase or lathering cleaning phase of a personal cleansing composition, is measured using a graduated cylinder and a tumbling apparatus. A 1,000 ml graduated cylinder is used which is marked in 10 ml increments and has a height of 14.5 inches at the 1,000 ml mark from the inside of its base (for example, Pyrex No. 2982). Distilled water (100 grams at 23° C.) is added to the graduated cylinder. The cylinder is clamped in a rotating device, which clamps the cylinder with an axis of rotation that transects the center of the graduated cylinder. One gram of the total personal cleansing composition (0.5 g of the first aqueous phase and 0.5 g of the second aqueous phase when measuring the total product, or 1 g of the first aqueous phase or second aqueous phase when the measuring the first aqueous phase or second aqueous phase only) is added into the graduated cylinder and the cylinder is capped. The cylinder is rotated at a rate of 10 revolutions in about 20 seconds, and stopped in a vertical position to complete the first rotation sequence. A timer is set to allow 30 seconds for the lather thus generated to drain. After 30 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells (“bubbles”) reach across the entire cylinder, the height at which at least 10 foam cells are required to fill the space is the first lather volume, also in ml up from the base. Foam cells larger than one inch in any dimension, no matter where they occur, is designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum foam height is 1,000 ml (even if the total foam height exceeds the 1,000 ml mark on the graduated cylinder). One minute after the first rotation is completed, a second rotation sequence is commenced which is identical in speed and duration to the first rotation sequence. The second lather volume is recorded in the same manner as the first, after the same 30 seconds of drainage time. A third sequence is completed and the third lather volume is measured in the same manner, with the same pause between each for drainage and taking the measurement.

The lather result after each sequence is added together and the Total Lather Volume determined as the sum of the three measurements, in ml. The Flash Lather Volume is the result after the first rotation sequence only, in ml, i.e., the first lather volume. Compositions according to the present invention perform significantly better in this test than similar compositions in conventional emulsion form.

Yield Point Method: A TA Instruments AR2000 Controlled Stress Rheometer can be used to determine the Yield Point of the second aqueous phase or the first aqueous phase. For purpose herein, the Yield Point is the amount of stress required to produce a strain of 1% on the liquid second aqueous phase or the first aqueous phase. The determination is performed at 25° C. with a 4 cm diameter serrated parallel plate measuring system and a 1 mm gap. The determination is performed via the programmed application of a logarithmic shear stress ramp (typically from about 0.1 Pa to about 1,000 Pa) over a time interval of 4 minutes. It is this amount of stress that results in a deformation of the sample, a shear stress vs. strain curve can be created. From this curve, the Yield Point of the liquid second aqueous phase can be determined. The liquid second aqueous phase or the first aqueous phase are measured either prior to combining in the composition, or after combining in the composition by separating the compositions by suitable physical separation means, such as centrifugation, pipetting, cutting away mechanically, rinsing, filtering, or other separation means.

Dialysis Method: The Dialysis Method is for determining the migration, or diffusion over time, of chemical components (particularly water) from one phase of a dual phase body wash to the other. It is designed for very viscous materials. Migration is accelerated using a specially designed cell with two chambers divided by a dialysis membrane. The bulk of the phases are kept separate but molecules smaller than 3,500 MW are free to diffuse. The high surface area to thickness ratio allows diffusion to go to equilibrium in a manageable time frame. The needed materials are a Migration cell Dialysis Membrane—regenerated cellulose, 3,500 MWCO, Pierce product no. 68035, cut open to lay flat, clamps, disposable syringes, and a flat-edged spatula

Loading and Unloading Test Materials into Cell: A first endplate made of Plexiglas™ having the dimensions of 6 inches in length, 5 inches in width and ½ inch depth is placed on a flat surface and topped with first gasket made of silicone rubber having same dimensions as end plate, with a cutout in the center that has the dimension of 4 inches in length by 1 ½ inches in width. The gasket is pressed down to form a seal with the endplate, then 20 grams of the first aqueous phase in a disposable syringe is dispensed into the space in the gasket. The dialysis membrane, having similar in dimensions to endplate and the first gasket, is placed on top of this and pressed down to form a seal with the first gasket. A second gasket made of the same material and same dimensions the first gasket is placed on top of the dialysis membrane and pressed down. The second aqueous phase is then dispensed into the space in the second gasket on top of the dialysis membrane. This is topped with the second endplate, having dimensions and made similar in materials as the first endplate, and the entire assembly is held together with clamps. It can be placed vertically on a flat surface for the duration of the test period. To remove the test materials, place the diffusion cell flat and disassemble in the reverse order, scraping each material out with a flat-edged spatula as it is exposed.

Analysis of Test Materials: Chloride ion is determined by titration with silver nitrate to a potentiometric endpoint. Moisture is determined using a variable temperature moisture analyzer such as the Mettler-Toledo HR73. A 0.5-1.0 g sample is heated with a 5 minute ramp to 140° C., switchoff mode 3.

Method of Use: The multi-phase personal cleansing compositions of the present invention are preferably applied topically to the desired area of the skin or hair in an amount sufficient to provide effective delivery of the skin cleansing agent and skin benefit agents to the applied surface. The compositions can be applied directly to the skin or indirectly via the use of a cleansing puff, washcloth, sponge or other implement. The compositions are preferably diluted with water prior to, during, or after topical application, and then subsequently rinsed or wiped off of the applied surface, preferably rinsed off of the applied surface using water or a water-insoluble substrate in combination with water. The present invention is therefore also directed to methods of cleansing the skin through the above-described application of the compositions of the present invention.

Method of Manufacture: The multi-phase personal cleansing compositions of the present invention may be prepared by any known or otherwise effective technique, suitable for making and formulating the desired multi-phase product form. It is effective to combine toothpaste-tube filling technology with a spinning stage design. Additionally, the present invention can be prepared by the method and apparatus as disclosed in U.S. Pat. No. 6,213,166 issued to Thibant, et al. The method and apparatus allows two or more compositions to be filled with a spiral configuration into a single container. The method requires that at least two nozzles be employed to fill the container. The container is placed on a static mixer and spun as the composition is introduced into the container.

Alternatively, it is effective to combine at least two phases by first placing the separate compositions in separate storage tanks having a pump and a hose attached. The phases are then pumped in predetermined amounts into a single combining section. Next, the phases are moved from the combining sections into the blending sections and the phases are mixed in the blending section such that the single resulting product exhibits a distinct pattern of the phases. The pattern is selected from the group consisting of striped, marbled, geometric, and mixtures thereof. The next step involves pumping the product that was mixed in the blending section via a hose into a single nozzle, then placing the nozzle into a container and filing the container with the resulting product. Specific non-limiting examples of such methods as they are applied to specific embodiments of the present invention are described in the following examples.

If the personal cleansing compositions contain stripes of varying colors it can be desirable to package these compositions in a transparent or translucent package such that the consumer can view the pattern through the package. Because of the viscosity of the subject compositions it may also be desirable to include instructions to the consumer to store the package upside down, on its cap to facilitate dispensing.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

### EXAMPLE

The following examples further describe and demonstrate embodiments within the scope of the present invention, given solely for the purpose of illustration and are not to be construed as limitations of the present invention, as many variations thereof are possible without departing from the spirit and scope of the invention. Table 1 described examples of the first aqueous phase of the multi-phase personal cleansing compositions of the present invention that are to be combined with the second aqueous phases described in Table 2.

TABLE 1
Examples of First Aqueous Phase

| Materials | Weight percent of Materials |
|---|---|
| Sodium Trideceth-3 Sulfate (ST3S) | 8.12 |
| Sodium Lauroamphoacetate (NaLaa) | 4.81 |
| Sodium Lauryl Sulfate (SLS) | 8.12 |
| Xanthan gum (Keltrol 1000 from Kelco) | 0.22 |
| Guar hydroxypropyltrimonium chloride | 0.6 |
| (NHance 3196 from Aqualon) | |
| Polyethylene glycol (Polyox WSR301 from | 0.15 |
| Dow) | |
| Tridecyl-3 alcohol (Iconol TDA3 from | 2 |
| BASF) | |
| Citric Acid | 0.65 (to pH 5.6) |
| Sodium Chloride | 4.75 |
| Preservative | 0.25 |
| Perfume | 2.0 |
| Water (Q.S.) | 68.3 |
| Salt molality ((wt % salt/MW)/(water | 1.44 |
| wt %)/1000) | |
| (includes the 1% salt that comes in with | |
| the Sodium Lauroamphoacetate) | |

The example in Table 1 can be prepared by conventional mixing techniques. Prepare the first aqueous by weighing water into a container and begin to agitate using an overhead mixer. Add the surfactants, ST3S, NaLaa and SLS, and agitate until smooth. Premix TDA-3, Polyox, and NHance 3196 and add to surfactants. Allow to mix until smooth. Add salt, and then the pH of the composition is adjusted with citric acid to pH 5.6. Add perfume. Transfer to sealed container. The T-Bar Viscosity of the Examples in Table 1 can be measured after 48 hours.

TABLE 2
Examples of Second Aqueous Phase comprising Polymer

| | Weight Percent of Materials | | | |
|---|---|---|---|---|
| | Comparative | Examples of the Present Invention | | |
| Materials | A | B | C | D |
| Stabylen 30 (acrylates vinyl- | 1 | 0.6 | 0.6 | 0.5 |
| isodecanoate crosspolymer from 3V) | | | | |
| Keltrol 1000 (xanthan gum from | 1 | 1.28 | 1.28 | 1 |
| Kelco) | | | | |
| Structure XL (hydroxyl propyl | | | | 2.5 |
| starch phosphate from National | | | | |
| Starch) | | | | |
| Polyox WSRN750 (poly- | | | 3 | |
| ethyleneglycol from Dow | | | | |
| Chemical) | | | | |
| Polyacrylic acid (Accusol 445 from | 0 | 3 | | 2 |
| Rhom and Haas) | | | | |
| Sodium Chloride | 7.5 | 7.5 | 7.5 | 7.5 |
| Preservative | 0.25 | 0.25 | 0.25 | 0.25 |
| Water | 90.5 | 87.6 | 87.6 | 86.5 |
| Sodium Hydroxide | To pH 5.6 | To pH 5.6 | To pH 5.6 | To pH 5.6 |
| Salt Molality ((wt %/MW)/(water | 1.42 | 1.46 | 1.46 | 1.48 |
| wt %/1000)) | | | | |

The examples of second aqueous phase A, B, C, and D shown in Table 2 can be prepared by conventional mixing techniques. Prepare the first aqueous by weighing water into a container and begin to agitate using an overhead mixer. Add Stabylen 30 and allow to hydrate until smooth, then add the Keltrol 1000 and allow to hydrate until smooth. For each of the batches the other polymers may then be added and allowed to hydrate until smooth. Add salt, pH adjust to 5.6 with sodium hydroxide, and then add preservative. Transfer to sealed container. The T-Bar Viscosity of the Examples in Table 1 can be measured after 48 hours.

The Dialysis Method as described above was performed on Examples from Table 1 and Table 2, and the results are shown in the following Table 3:

TABLE 3
Dialysis Results

| | Comparative | | | |
|---|---|---|---|---|
| | Example | Examples of the Present Invention | | |
| | Example from | Examples from | Examples from | Examples from |
| Initial weight | Table 1 and | Table 1 and | Table 1 and | Table 1 and |
| percent of the | Example A in | Examples B in | Examples C in | Examples D in |
| Phase = 68.3 | Table 2 | Table 2 | Table 2 | Table 2 |
| Final water | 73 | 68 | 70 | 67.4 |
| weight percent | | | | |
| Delta = Change | 4.7 | −0.3 | 1.7 | −0.9 |
| in weight % of | | | | |
| water Initial and Final | | | | |
| Percent water | 6.9% | 0.4% | 2.5% | 1.3% |
| weight change | | | | |
| (absolute value | | | | |
| of Delta/Initial | | | | |
| weight percent) | | | | |

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-phase personal cleansing composition comprising
a. a first aqueous phase comprising a surfactant, a salt and water; and
b. a second aqueous phase comprising a polymer, a salt and water;
wherein the molality of the salt of the first aqueous phase differs from that of the salt of the second aqueous phase by less than about 6%;
wherein the weight percent of water of the first aqueous phase differs by at least 5% from that in the second aqueous phase;
wherein the viscosity of the first aqueous phase differs from that of the second aqueous phase by less than 25%; and
wherein the first aqueous phase and the second aqueous phase are packaged in physical contact with one another.

2. The personal cleansing composition of claim 1, wherein the polymer of the second aqueous phase is a high solution viscosity polymer having a 1% viscosity at pH 7 of greater than 750 centipose.

3. The personal cleansing composition of claim 1, wherein the second aqueous phase further comprises an anioinic polymer.

4. The personal cleansing composition of claim 1, wherein the second aqueous phase further comprises a linear homopolymer.

5. The personal cleansing composition of claim 1, wherein the second aqueous phase further comprises a linear polyacrylic acid.

6. The multi-phase personal cleansing composition of claim 1, where the second aqueous phase further comprises a cross-linked polyacrylic acid.

7. A multi-phase personal cleansing composition comprising
a. a first aqueous phase comprising a surfactant, a salt and water; and
b. a second aqueous phase comprising at least one high solution viscosity polymer having a 1% viscosity at pH 7 of greater than 750 centipose, a salt and water;
wherein the molality of the salt in the first aqueous phase differs from that of the salt of the second aqueous phase by less than about 6%;
wherein the weight percent of water of the first aqueous phase differs by at least 5% from that of the second aqueous phase;
wherein the viscosity of the first aqueous phase differs from that of the second aqueous phase by less than 25%; and
wherein the first aqueous phase and second aqueous phase are packaged in physical contact with one another.

8. The multi-phase personal cleansing composition of claim 7, wherein the first aqueous phase comprises less than about 3%, by weight of the first aqueous phase, of the surfactant.

9. The personal cleansing composition of claim 7 wherein the second aqueous phase further comprises a linear ionic polymer with a 1% viscosity at pH 7 less than 500 centipose.

10. The personal cleansing composition of claim 7, wherein the second aqueous phase further comprises an anioinic polymer.

11. The personal cleansing composition of claim 7, wherein the second aqueous phase further comprises a linear homopolymer.

12. The personal cleansing composition of claim 7, wherein the second aqueous phase further comprises a linear polyacrylic acid.

13. The multi-phase personal cleansing composition of claim 7, where the second aqueous phase further comprises a cross-linked polyacrylic acid.

14. The multi-phase personal cleansing composition of claim 7, wherein the first aqueous phase further comprises a polymer.

15. The multi-phase personal cleansing composition of claim 13, wherein the first aqueous phase comprises a lesser weight percent of the polymer than the weight percent of the polymer in the second aqueous phase.

16. The multi-phase personal cleansing composition of claim 7, wherein the second aqueous phase further comprises a surfactant.

17. The multi-phase personal cleansing composition of claim 16, wherein the second aqueous phase comprises a weight percent of salt greater than a weight percent of surfactant.

18. The multi-phase personal cleansing composition of claim 7 wherein the first aqueous phase is a lamellar phase.

19. The multi-phase personal cleansing composition of claim 7, wherein the first aqueous phase has a percent water weight change of less than 5% after storage in physical contact with the second aqueous phase for 7 days according to the Dialysis Method.

20. The multi-phase personal cleansing composition of claim 7, wherein the first aqueous phase comprises a viscosity greater the second aqueous phase.
